(19) United States
(12) Patent Application Publication
(10) Pub. No.: US 2012/0045429 A1
(43) Pub. Date: Feb. 23, 2012

(54) Methods and Agent for Modulating the RNA Polymerase II-Histone Surface

(75) Inventors: Vasily Studitsky, Edison, NJ (US); Olga Studitskaia, Edison, NJ (US); Daria Gaykalova, Highland Park, NJ (US)

(73) Assignee: University of Medicine and Dentistry of New Jersey, Somerset, NJ (US)

(21) Appl. No.: 12/952,295

(22) Filed: Nov. 23, 2010

Related U.S. Application Data
(60) Provisional application No. 61/374,448, filed on Aug. 17, 2010.

(51) Int. Cl. A61K 39/395; C07K 2/00; C07K 16/00; C07H 21/00; C07C 53/00; C07J 1/00; C07D 473/00; C07D 239/00; A61K 38/02; A61K 31/7052; A61K 31/70; A61K 31/20; A61K 31/56; A61K 31/52; A61K 31/505; A61P 43/00; C12Q 1/68

(52) U.S. Cl. 424/130.1; 435/6.1; 530/300; 530/387.1; 536/23.1; 536/1.11; 554/1; 552/502; 544/264; 544/242; 514/1.1; 514/44 R; 514/23; 514/558; 514/169; 514/263.1; 514/256

(57) ABSTRACT

The present invention relates to the identification of an intranucleosomal DNA loop formed during transcription through a nucleosome and use of the same to identify an agent that modulates the RNA Polymerase II-histone surface.

### INTRODUCTION

This application claims benefit of priority to U.S. Provisional Application Ser. No. 61/374,448, filed Aug. 17, 2010, the content of which is incorporated herein by reference in its entirety.

This invention was made with government support under grant number NSF 0549593 awarded by the National Science Foundation and grant numbers RO1 GM58650 and RO1 GM067153 awarded by the National Institutes of Health. The government has certain rights in the invention.

### BACKGROUND OF THE INVENTION

Chromatin structure tightly compacts DNA in eukaryotic nucleus, yet allows regulated access of proteins to DNA and enables efficient progression of DNA and RNA polymerases along the template. Efficient maintenance and recovery of nucleosomal organization during and after passage of the RNA Polymerase II (Pol II) transcription complex is essential for proper gene regulation and cell survival (Martens, et al. (2005) Genes Dev. 19:2695-2704). Recovery of chromatin structure occurs through two different mechanisms during moderate and intense transcription, respectively (Thiriet & Hayes (2006) Results Probl. Cell Differ. 41:77-90; Kulaeva, et al. (2007) Mutat. Res. 618:116-129). During very intense transcription, nucleosome structure can be disrupted, wherein partial loss (Kristjuhan & Svejstrup (2004) EMBO J. 23:4243-4252; Lee, et al. (2004) Nat. Genet. 36:900-905; Schwabish & Struhl (2004) Mol. Cell. Biol. 24:10111-10117; Petesch & Lis (2008) Cell 134:74-84; Zhao, et al. (2005) Mol. Cell. Biol. 25:8985-8999) and exchange (Wirbelauer, et al. (2005) Genes Dev. 19:1761-1766; Schwartz & Ahmad (2005) Genes Dev. 19:804-814; Thiriet & Hayes (2005) Genes Dev. 19:677-682; Dion, et al. (2007) Science 315:1405-1408; Rufiange, et al. (2007) Mol. Cell 27:393-405; Jamai, et al. (2007) Mol. Cell 25:345-355) of all core histones at the transcribed regions of genes have been reported. In contrast, nucleosomes remain associated with genes transcribed with lower efficiency (Kristjuhan & Svejstrup (2004) supra; Lee, et al. (2004) supra; Schwabish & Struhl (2004) supra). On moderately transcribed genes, fast and extensive transcription-dependent exchange of H2A/H2B, but not H3/H4, histones was observed (Wirbelauer, et al. (2005) supra; Schwartz & Ahmad (2005) supra; Thiriet & Hayes (2005) supra; Dion, et al. (2007) supra; Rufiange, et al. (2007) supra; Jamai, et al. (2007) supra).

The Pol II-type mechanism of transcription through chromatin in vitro is characterized by three features that are conserved from yeast to human (Bondarenko, et al. (2006) Mol. Cell 24:469-479): (i) A high nucleosomal barrier to transcription, which Pol II alone can overcome only at 300 mM or higher ionic strength (Bondarenko, et al. (2006) supra; Izban & Luse (1991) Genes Dev. 5:683-696; Kireeva, et al. (2002) Mol. Cell 9:541-552); (ii) The displacement of a single H2A/H2B dimer (Kireeva, et al. (2002) supra; Belotserkovskaya, et al. (2003) Science 301:1090-1093; Angelov, et al. (2006) EMBO J. 25:1669-1679) that matches the apparent effect of Pol II passage in vivo (Thiriet & Hayes (2005) supra; Kimura & Cook (2001) J. Cell. Biol. 153:1341-1353); (iii) The subnucleosome (DNA-bound histone hexamer formed upon release of H2A/H2B dimer from the octamer) survives Pol II passage through a nucleosome and remains at the original position on DNA (Kireeva, et al. (2002) supra). A considerably different, Pol III-type mechanism, which involves transfer of a complete histone octamer from in front of the transcribing enzyme to behind it is used by Pol III and single-subunit bacteriophage RNA polymerase (RNAP) (Studitsky, et al. (1994) Cell 76:371-382; Studitsky, et al. (1995) Cell 83:19-27; Studitsky, et al. (1997) Science 278:1960-1963).

It has been shown that nucleosomes positioned on DNA sequences having a high affinity to histones (HA sequences) present a polar barrier to transcription by Pol II in vitro (Bondarenko, et al. (2006) supra). In one (non-permissive) orientation, the nucleosomal barrier is high, whereas in the opposite (permissive) orientation, as well as in nucleosomes that lack the HA sequences, the height of the nucleosomal barrier is much lower.

### SUMMARY OF THE INVENTION

The present invention features a method for identifying an agent that modulates the RNA Polymerase II-histone surface by contacting a nucleosome with a test agent in the presence of a RNA Polymerase II elongation complex and determining whether the agent modulates resolution of a Ø-loop formed by the nucleosome and RNA Polymerase II elongation complex. Agents that stabilize or destabilize the RNA Polymerase II-histone surface are also provided as are methods of using such agents in methods for preventing or treating disease.

### BRIEF DESCRIPTION OF THE DRAWINGS

FIG. 1 shows that Promoter-distal high-affinity nucleosome positioning sequences dictate the high nucleosomal barrier to transcription by Pol II. Pol II paused at the +45 region (step 1) could form a Ø-loop and sterically displace the promoter-distal DNA end from the octamer (steps 2 and 3) to facilitate further transcription through a nucleosome. Strong histone-DNA contacts in the R3 high-affinity region would block DNA displacement and transcription. Nucleosomal DNA is stippled and the histone octamer is shown diagonal stripe. The direction of transcription is indicated by an arrow.

FIG. 2 illustrates the Pol II-type mechanism of chromatin remodeling. As RNAP approaches a nucleosome (step 1; small arrows indicate direction of transcription), upstream nucleosomal DNA is partially uncoiled (dashed arrows) from the octamer (step 2). Then a transient Ø-loop is formed at position +49 (step 3), and RNAP displaces the promoter-distal end of nucleosomal DNA (step 4). As RNAP continues transcription, the DNA-histone contacts upstream of the enzyme serve as an anchor to recover the nucleosome behind RNAP (step 5). Below, the mechanism of nucleosome survival during transcription. Only about two-thirds of a DNA supercoil on the surface of the octamer is shown.

FIG. 3 shows that removal of the promoter-proximal H2A-H2B dimer results in Pol II arrest in the +45 region of the nucleosome. During Pol II progression through +45 region, the Ø-loop is formed (step 1). Removal of the promoter-distal D-dimer promotes dissociation of the D end of nucleosomal DNA (step 2) and further transcription (step 3), and thus would decrease the height of the +45 barrier. In contrast, removal of the proximal P-dimer favors release of the P end of nucleosomal DNA (2′), thereby eliminating upstream DNA-histone contacts and destabilizing the Ø-loop, an intermediate thought to facilitate transcription. Thus, the P-dimer removal is expected to increase the height of the +45 barrier considerably.

FIG. 4 depicts a feedback mechanism that allows survival of histones H3 and H4 during Pol II transcription. The feedback mechanism depicted in FIG. 4A indicates that as Pol II approaches a nucleosome (step 1), its progression is initially accompanied by displacement of DNA behind the enzyme (step 2) and could result in displacement of the histone octamer into solution (step 3). However, when Pol II reaches the +45 region of strong DNA-histone interactions, the Ø-loop is formed (step 4). Ø-loop formation prevents octamer displacement and allows further transcription by facilitating DNA displacement in front of Pol II (steps 4 and 5). Thus, Pol II transcription is coupled with nucleosome survival. As shown in FIG. 4B, Pol II-type mechanism allows survival of H3 and H4 histones on DNA during transcription. Transcription by the Pol III-type mechanism would induce backward nucleosome translocation and displacement and exchange of all core histones. In contrast, during transcription by Pol II, nucleosomes are not translocated, and only H2A-H2B histones are exchanged. Thus, specifically modified H3 and H4 histones could survive Pol II transcription.

FIG. 5 shows that nucleosomes containing Sin mutant histones are more likely to dissociate during pol II transcription. DNA-labeled nucleosomes containing wild-type or Sin mutant histones were transcribed by yeast pol II at KCl concentrations of 40, 150 and 300 mM; intact complexes were then resolved from free DNA by native polyacrylamide gel electrophoresis. For the no-chase control (nc), transcription was performed in the absence of UTP and pol II was stalled upstream of the nucleosome. The graph shows the amounts of histone-free DNA produced after transcription at 150 mM KCl. Amounts were quantified and normalized to the overall amount of active ECs. H3, histone 3; H4 histone 4.

### DETAILED DESCRIPTION OF THE INVENTION

Maintenance of proper genetic information (DNA sequence) and epigenetic and regulatory marks (DNA and histone modifications) during various cellular processes (e.g., DNA replication and transcription) is essential for cell viability and normal functioning. In particular, defects in enzymes involved in maintenance of epigenetic marks during transcription by RNA polymerase II (Pol II) (e.g., SET-type histone methyltransferases) lead to development of numerous aggressive forms of human cancer (leukemias). Furthermore, mutations in proteins that maintain epigenetic marks during transcription lead to increased DNA instability. Thus, maintenance of epigenetic marks during transcription by Pol II is important for maintenance of DNA stability and cell functioning.

It has now been found that maintenance of epigenetic marks during transcription critically depends on formation of a distinct Pol II-histone complex. Moreover, this analysis identified the surface for protein-protein interactions that allows formation of this key complex. Therefore, this protein-protein surface provides a target for development of drugs targeted to prevent, treat and/or cure various human diseases including cancers.

Thus, the present invention relates to the maintenance of chromatin structure and histone marks during transcription and to targets and methods for the development of drugs targeted to prevent, treat and/or cure various human diseases including cancers. More specifically, the invention provides drugs that stabilize the Pol II-histone surface to correct abnormalities caused by improper maintenance of histones, or modifications thereof, or prevent development of such abnormalities. In addition, the invention provides drugs that destabilize the Pol II-histone surface to correct abnormalities caused by over efficient maintenance of histones, or modifications thereof, or prevent development of such abnormalities. Since the critical identified protein-protein surface is likely to be involved in other processive cellular processes, in addition to transcription (e.g., ATP-dependent remodeling, DNA repair, DNA recombination and DNA replication), drugs targeting the histone surface could target these processes.

Accordingly, the present invention features a method for identifying an agent that modulates the RNA Pol II-histone surface by contacting a nucleosome with a test agent in the presence of a RNA Pol II elongation complex and determining whether the agent modulates the resolution of the Ø-loop formed by the nucleosome and RNA Polymerase II elongation complex. As is known in the art, a nucleosome is an approximately 146-147 bp segment of DNA wrapped around a histone octamer composed of pairs of each of the four core histones (H2A, H2B, H3, and H4). The chromatin fiber is further compacted through the interaction of a linker histone, H1, with the DNA between the nucleosomes to form higher order chromatin structures. Nucleosomes can be isolated from natural sources or, in accordance with particular embodiments, reconstituted using purified and isolated components (i.e., histones and DNA).

Histones of use in accordance with the present invention can be from any eukaryotic species and in particular embodiments are from a mammal such as a human, mouse, dog, rat, pig, etc. Moreover, the octamer can be composed of histones from one species, or alternatively from more than one species, i.e., a hybrid octamer.

TABLE 1

| Source | Histone | GENBANK Accession No. |
|---|---|---|
| Homo sapiens | H2A | NP_734466 |
| | H2B | NP_733759 |
| | H3 | NP_003484 |
| | H4 | NP_003539 |
| Mus musculus | H2A | NP_835736 |
| | H2B | NP_075911 |
| | H3 | NP_032236 |
| | H4 | NP_291074 |
| Rattus norvegicus | H2A | NP_068612 |
| | H2B | NP_072173 |
| | H3 | NP_446437 |
| | H4 | NP_073177 |

Histones can be purified from eukaryotic cells (i.e., “native” histones) or may be recombinantly produced using any conventional eukaryotic or prokaryotic expression system. Such systems are well-known and routinely employed in the art. Moreover, commercial sources such as INVITROGEN, CLONTECH, STRATAGENE and PROMEGA provide a variety of different vectors and host cells for producing recombinant proteins, with and without tags (e.g., glutathione-S-transferase, FLAG, His6, etc.). The recombinant protein thereafter is purified from contaminant soluble proteins and polypeptides using any of the following suitable purification procedures: by fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; gel filtration using, for example, SEPHADEX G-75; ligand affinity chromatography, and protein A SEPHAROSE columns to remove contaminants such as IgG.

In addition to recombinant production, a protein of the invention may be produced by direct peptide synthesis using solid-phase techniques (Merrifield (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer, Boston, Mass.). Various fragments of a protein of the invention may be chemically-synthesized separately and combined using chemical methods to produce a full-length molecule (He, et al. (2003) Proc. Natl. Acad. Sci. USA 100(21):12033-8; Shogren-Knaak and Peterson (2004) Methods Enzymol. 375:62-76).

Once the core histones are produced and/or isolated, they are mixed with a DNA molecule, preferably containing a nucleosome positioning sequence as described herein, under appropriate conditions so that nucleosomes are formed. In some embodiments, the mixture can further contain histone H1. In other embodiments, the nucleosome positioning sequence has the nucleotide sequence of SEQ ID NO:7 or SEQ ID NO:8. In further embodiments, the nucleosome positioning sequence and/or histone have one or mutations associated with a disease.

In particular embodiments, the nucleosomes of the present invention are immobilized. Immobilization, for the purposes of the present invention, means that the nucleosomes are covalently or non-covalently attached to a matrix or solid support. Such solid supports include beads, microtiter plates and the like. By way of illustration, glutathione-S-transferase tagged histones can be adsorbed onto SEPHAROSE beads (Sigma Chemical, St. Louis, Mo.) or glutathione-derivatized microtiter plates to immobilize the nucleosome. Alternatively, the DNA molecule of the nucleosome can be tagged, e.g., as disclosed herein and used to immobilize the nucleosome.

As with the histones, the RNA Pol II elongation complex can be isolated and purified from a variety of natural sources or alternatively reconstituted using conventional methods. See, e.g., Kettenberger, et al. (2004) Mol. Cell 16:955-965.

In carrying out the method of the present invention, a test agent is added to a point of application, such as a microtiter well, containing nucleosomes and RNA Pol II elongation complex. It is contemplated that the agent can be added before or after formation of the Ø-loop by nucleosomes and RNA Pol II elongation complexes. Agents which can be screened in accordance with the instant assay can be rationally designed from crystal structure information or identified from a library of test agents. Test agents of a library can be synthetic or natural compounds. A library can comprise either collections of pure agents or collections of agent mixtures. Examples of pure agents include, but are not limited to, peptides, polypeptides, antibodies, oligonucleotides, carbohydrates, fatty acids, steroids, purines, pyrimidines, lipids, synthetic or semi-synthetic chemicals, and purified natural products, derivatives, structural analogs or combinations thereof. Examples of agent mixtures include, but are not limited to, extracts of prokaryotic or eukaryotic cells and tissues, as well as fermentation broths and cell or tissue culture supernatants. In the case of agent mixtures, one may not only identify those crude mixtures that possess the desired activity, but also monitor purification of the active component from the mixture for characterization and development as a therapeutic drug. In particular, the mixture so identified can be sequentially fractionated by methods commonly known to those skilled in the art which may include, but are not limited to, precipitation, centrifugation, filtration, ultrafiltration, selective digestion, extraction, chromatography, electrophoresis or complex formation. Each resulting subfraction can be assayed for the desired activity using the original assay until a pure, biologically active agent is obtained.

Agents of interest in the present invention are those with functional groups necessary for structural interaction with proteins and/or nucleic acids, particularly hydrogen bonding, and typically include at least an amine, carbonyl, hydroxyl or carboxyl group. The agents often include cyclical carbon or heterocyclic structures and/or aromatic or polyaromatic structures substituted with one or more of the above functional groups.

Subsequent to applying the test agent to the nucleosome and Pol II elongation complex, it is determined whether the test agent modulates the resolution of the Ø-loop formed by the nucleosome and RNA Polymerase II elongation complex. As described herein, a Ø-loop is an intranucleosomal DNA loop or “zero-size” loop, which is resolved by Pol II transcription of the DNA. Resolution of the Ø-loop can be determined by methods described herein or another suitable technique, e.g., monitoring presence or rate of transcription. It is contemplated that agents identified in accordance with the present assay can either activate, stimulate or stabilize Ø-loop formation or inhibit, block or destabilize the Ø-loop. Accordingly, the term “modulating” or “modulates” is intended to encompass both activators/stabilizers and inhibitors/destabilizers. Given their use in the treatment of diseases such as cancer, particular embodiments of the present invention embrace agents that activate or stabilize Ø-loop formation.

An agent identified in accordance with the instant assay method can be formulated into a pharmaceutically acceptable composition for therapeutic use, e.g., in the treatment of cancer. The agent can be formulated with any suitable pharmaceutically acceptable carrier or excipient, such as buffered saline; a polyol (e.g., glycerol, propylene glycol, liquid polyethylene glycol and the like); carbohydrates such as glucose, mannose, sucrose or dextrans, mannitol; amino acids such as glycine; antioxidants; chelating agents such as EDTA or glutathione; preservatives or suitable mixtures thereof. In addition, a pharmaceutically acceptable carrier can include any solvent, dispersion medium, and the like which may be appropriate for a desired route of administration of the composition. The use of sustained-release delivery systems such as those disclosed by Silvestry, et al. ((1998) Eur. Heart J. 19 Suppl. I:I8-14) and Langtry, et al. ((1997) Drugs 53(5):867-84), for example, are also contemplated. The use of such carriers for pharmaceutically active substances is known in the art. Suitable carriers and their formulation are described, for example, in Remington: The Science and Practice of Pharmacy, Alfonso R. Gennaro, editor, 20th ed. Lippincott Williams & Wilkins: Philadelphia, Pa., 2000.

The route of administration of a composition containing an agent identified herein will depend, in part, on the chemical structure of the molecule. For example, polypeptides and polynucleotides, for example, are not efficiently delivered orally because they can be degraded in the digestive tract. However, methods for chemically modifying polypeptides, for example, to render them less susceptible to degradation by endogenous proteases or more absorbable through the alimentary tract may be used (see, for example, Blondelle, et al. (1995) Trends Anal. Chem. 14:83-92; Ecker & Crook (1995) BioTechnology 13:351-360

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the composition to treat a pathologic condition in a subject depends on many factors including the age and general health of the subject as well as the route of administration and the number of treatments to be administered. In view of these factors, the skilled artisan would adjust the particular dose as necessary. In general, the formulation of the composition and the routes and frequency of administration are determined, initially, using Phase I and Phase II clinical trials.

A central characteristic of cancer is deregulation of transcription control leading to activation of expression of growth-promoting genes, as well as silencing of genes with the tumor suppressor functions. Importantly, mutations found in tumor cells cannot alone explain the complexity of the change in pattern of gene expression. Epigenetic changes in the transcribed regions, such as DNA methylation, covalent modifications of the histones, and ATP-dependent chromatin remodeling have been recently identified as key universal components of the transformation process. It has been suggested that chromatin remodeling and modification occurs during promoter activation, and is associated with transcription elongation. Based upon the data presented herein, inefficient resolution of the Ø-loop can cause strong nucleosome-specific pausing, and the paused intermediate could be a target for regulation of the rate of elongation through chromatin. Accordingly, agents identified by the screening assay of the present invention find application in modulating the effects of epigenetic changes by stabilizing or destabilizing RNA Pol II-histone surfaces. While the present invention is of particular use in the prevention and/or treatment of human cancers, other diseases could also be caused by defects in maintenance of histones and their modifications (epigenetic and regulatory marks) during Pol II transcription. For example, genetic mutations affecting the protein-protein surface are likely to be involved in numerous human diseases and may also provide a foundation for diagnostics of these diseases.

The invention is described in greater detail by the following non-limiting examples.

### Example 1

### Mechanism of Chromatin Remodeling and Recovery During Passage of RNA Polymerase II

Protein Purification Methods. Hexahistidine-tagged E. coli RNAP, Pol II, core histones and GreB protein were purified using published protocols (Kireeva, et al. (2002) supra; Artsimovitch, et al. (2003) J. Biol. Chem. 278:12344-12355; Vassylyeva, et al. (2007) EMBO Rep. 8:1038-1043).

DNA Templates and Sequence Alignment. The 603 and 603R templates for Pol II are known in the art (Bondarenko, et al. (2006) supra). The variants of the 603R template (603R, 603R-L and -R) were prepared by annealing pairs of long overlapping oligonucleotides and filling-in with the Klenow fragment of DNA polymerase I (NEB). Then the double-stranded DNA fragments were PCR-amplified using a different pair of primers to obtain 201-bp fragments. The 201-bp fragments were gel-purified and PCR-amplified with another pair of primers to obtain 262-bp fragments containing TspR1 site. After digestion with TspRI (NEB) 249-bp fragments were obtained. The 110-bp DNA fragments for reconstitution of hexasomes and tetrasome were obtained by PCR-amplification of the plasmid pGEM-3Z/603 (Thastrom, et al. (2004) J. Mol. Biol. 338:695-709) with different pairs of primers, followed by TspR1 digestion.

To obtain the 603-42 and 603-49 templates for Pol II, the original 603 template was mutated at four or six positions to allow stalling of Pol II at +42 or +49 positions in the 603 nucleosome. The nucleosome positioning sequences were amplified by PCR and digested with TspRI (NEB) to obtain the 149-bp DNA fragment.

Protocol for Reconstitution of Nucleosomes and Subnucleosomes. The 149-bp and 249-bp DNA fragments were gel-purified and used for nucleosome reconstitution by octamer exchange at 1:3 DNA:chromatin ratio (Kireeva, et al. (2002) supra). The hexasomes were reconstituted using chicken erythrocytes core histones by dialysis from 2 M NaCl (Studitsky (1999) Methods Mol. Biol. 11:17-26).

To obtain 603-42 and 603-49 templates for E. coli RNAP the original 603 template was mutated to replace four or six nucleotides in DNA and allow stalling of RNAP at the +42 or +49 positions within the 603 nucleosome, respectively. The nucleosome positioning sequences were amplified by PCR, digested by TspRI (NEB) and ligated through the TspRI site to a T7A1 promoter-bearing fragment (Walter, et al. (2003) J. Biol. Chem. 278:36148-36156). Ligated products were re-amplified with one 5′-end-labeled primer, gel-purified, and assembled into nucleosomes. Nucleosomes were reconstituted on the DNA templates by histone octamer transfer from chicken -H1 erythrocyte donor chromatin (Kireeva, et al. (2002) supra).

Transcription of Nucleosomes and Subnucleosomes. E. coli RNAP: Elongation complexes containing 11-mer RNA (EC−39) were formed on pre-assembled nucleosomal templates as described (Kettenberger, et al. (2004) Mol. Cell 16:955-965). In experiments with labeled RNA EC−39 was pulse-labeled in the presence of [α-³²P]-GTP (3000 Ci/mmol, PerkinElmer Life Sciences). Then EC−39 was extended in the presence of a subset of NTPs to form EC−5. In footprinting experiments, all steps were performed in solution. In experiments involving labeled RNA and GreB treatment, EC−5 was immobilized on Ni-NTA-agarose (Walter, et al. (2003) supra). After extensive washes the complexes were eluted from Ni-NTA beads in the presence of 100 mM imidazole and transcription was continued in solution. EC−5, EC+41 or EC+49 were formed in the presence of 1 μM ATP on the 603-42 or 603-49 templates. EC−5 was further extended in the presence of 300 μM CTP, UTP, GTP and 150 μM 3′ dATP at 25° C. for 4 minutes or in the presence of 200 μM of all NTPs in TB300. Labeled RNA was purified and separated by denaturing PAGE. Transcription by Pol II was performed as described (Kireeva, et al. (2002) supra; Walter, et al. (2003) supra).

DNaseI Footprinting Methodology. DNase I footprinting was carried out at a final concentration of end-labeled templates of 2.5 μg/ml in the presence of 10-fold weight excess of unlabeled -H1 chicken erythrocyte chromatin in TB100 (20 mM Tris HCl pH 8.0, 5 mM MgCl₂, 2 mM β-ME, 100 mM KCl). DNaseI was added to a final concentration 20-50 U/ml for 30 seconds at 37° C. after formation of the desired ECs. The reactions were terminated by adding EDTA to 10 mM. The samples were resolved in a native gel (Kireeva, et al. (2002) supra). Gel fragments containing desired complexes were cut, DNA extracted, purified and analyzed by denaturing PAGE. The gels were quantified using a PHOSPHORIMAGER.

Analysis of Pol II Elongation Complexes. To stall Pol II at +42 and +49 positions in the 603 nucleosome, EC−39 was assembled and immobilized on Ni-NTA-agarose (Kireeva, et al. (2002) supra). Then EC−39 was ligated for 2 hours at 16° C. in TB40 to nucleosomes assembled on the 149-bp DNA fragment, washed, eluted into solution (Kireeva, et al. (2002) supra) and extended in the presence of a subset of NTPs to form EC−5, EC+41 or EC+49 in TB300 at 20° C. The complexes were diluted 3-fold with TB0 to decrease concentration of KCl to 100 mM, digested with the restriction enzymes indicated, and separated by native PAGE. The gels were quantified using a PHOSPHORIMAGER.

Modeling of Pol II at the Position +39 in a Nucleosome. The model was built manually using the O program (Jones, et al. (1991) Acta Crystallogr. A. 47 (Pt 2), 110-119) and the structure of nucleosome (PDB ID 1aoi; Luger, et al. (1997) Nature 389:251-260) to which at the first step the high resolution structure of the bacterial EC (PDB ID 2o5i; Vassylyev, et al. (2007) Nature 448:157-162) was docked. A 50-bp DNA region was displaced from the octamer surface starting from the end of nucleosomal DNA downstream of the EC to allow formation of the Ø-loop. The 30 bp of nucleosomal DNA was also removed at and around the position of the active center of the enzyme (+39), including upstream and downstream sequences that constitute the transcription bubble buried within the RNAP structure (Ø-loop). The bacterial enzyme revealed no significant steric clashes with the nucleosome. On the next step, the structure of the yeast Pol II (Kettenberger, et al. (2004) Mol. Cell 16:955-965) was modeled on the nucleosome through superposition of the bacterial RNAP and Pol II backbones. In the model, structural configuration of the nucleic acids in the transcription bubble observed in the experimental structures of the bacterial and eukaryotic ECs remained nearly intact. The structure of the histone core was not modified except for truncation of several N-terminal histone tails facing RNAP to avoid steric hindrance with the enzyme. In the complex with RNAP, these protruding flexible tails may readily adopt drastically distinct conformations as compared to the intact nucleosome structure; therefore modeling of the positions of histone tails was not feasible. The only notable (but still quite subtle) alterations, in which several Pol II protruding structural domains on the surface were slightly rotated as the rigid bodies (by ˜35-40° to avoid close contacts with the neighboring histone fragments, resulted in opening of the Pol II “jaws” and, subsequently, in a slight widening (4-5 Å) of the main cavity. Similar alterations have been observed in previously described Pol II structures (Cramer, et al. (2001) Science 292:1863-1876; Cramer (2002) Curr. Opin. Struct. Biol. 12:89-97; Cramer, et al. (2000) Science 288:640-649).

Analysis of the Structural Features of the Modeled Intranucleosomal Pol II EC+39. The molecular electrostatic surfaces of the proteins were calculated and displayed by PyMOL script. The sequences of S. cerevisiae Pol II largest subunit (RPB1) and E. coli RNAP β′ subunit were aligned by NCBI BLAST by composition matrix adjustment method.

Quantitation of Sensitivity of Pol II ECs to Restriction Enzymes. Bands on the gels were quantified using a PHOSPHORIMAGER. For the Cac8I digestion, the amounts of digested DNA present in experimental samples were normalized using the control digestion. The inverse ratios of the amounts of normalized digested DNA to total amounts of the active ECs were calculated. For the StyI digestion, the inverse ratios of the amounts of digested active ECs to total amounts of the active ECs were calculated.

Asymmetric Distribution of the HA Sequences Dictates the Polarity of the Nucleosomal Barrier to Transcription. It was first evaluated whether the polar barrier to transcription by Pol II (Bondarenko, et al. (2006) supra; Hall, et al. (2009) Nat. Struct. Mol. Biol. 16:124-129) is dictated by asymmetric location of the HA DNA sequences within the nucleosome. Well-characterized 601, 603 and 605 nucleosome positioning sequences were aligned according to hydroxyl radical footprinting data and compared with the HA consensus sequence (Thastrom, et al. (2004) supra). The sequences were: 601, 5′-GAGTAATCCCCTTGGCGGTTAAAACGCGGGGGACAGCGCGTACGTGCGTTTAAGCGGTG CTAGAGCTGTCT-3′ (SEQ ID NO:1); 603R, 5′-TTCAACATCGATGCACGGTGGTTAGCCTTGGATTGCGCTCTACCGTGCGCTAAGCGTAC TTAGAAGCCCGA-3′ (SEQ ID NO:2); 605R 5′-TGAATACCCTTGGGCGGCTAAAACGACGGGGCTAGGGTTGTAACGTCGTTTAAGCGTAT CTAGACCGGTCT-3′ (SEQ ID NO:3); and HA consensus, 5′-AXXXXXXTCTAGXXXXGCTTAAAXXGXXXXAXAXGXCXTXTXXXXCXXTTTAAGCXXXX CTAGAXXXXXXT-3′ (SEQ ID NO:4), wherein underlined sequences show consensus. In all cases, the consensus-like sequences were localized highly asymmetrically: there was considerably higher similarity to the consensus within the promoter-distal half of nucleosomal DNA in the non-permissive orientations than in the permissive orientations (88, 65 and 88% vs. 47, 35 and 59% for the 601, 603 and 605 templates, respectively.

To dissect the effects of different sequence elements on their affinity for core histones and on nucleosome positioning, various regions of the 603R template were mutated and the properties of resulting nucleosomes were analyzed by native PAGE. Mutations in the consensus-like sequences in the promoter-proximal, left (-L) half of the template 603R-L (TCGATGCACGGTGGTTAGCCTTGGATTG (SEQ ID NO:5), underlined residues indicate location of mutations) barely affected the affinity or the positioning properties of the 603R template. Similar changes introduced in the right half (603R-R template; TCTACCGTGCGCTAAGCGTACTTAGA (SEQ ID NO:6), underlined residues indicate location of mutations) decreased the affinity of the template for core histones (as indicated by the appearance of histone-free DNA in the gel) and resulted in the loss of nucleosome positioning. Thus sequences located in the distal half of the 603R template dictate both DNA-histone affinity and nucleosome positioning. Partial mutagenesis of the right half (603R-R(2-3) template; TAAGCGTACTTAGA (SEQ ID NO:7)) also resulted in decreased affinity of the template for core histones, but did not interfere with nucleosome positioning. Thus, the determinants for nucleosome positioning and DNA-histone affinity are distinct.

The polar distribution of the HA sequences in the nucleosomal DNA may determine the transcriptional polarity observed previously. To evaluate this, nucleosomes formed on the 603R sequence and its variants were transcribed by Pol II. The experiments were conducted using mononucleosomal templates, which recapitulate many important aspects of the mechanism of chromatin transcription by Pol II in vivo (Bondarenko, et al. (2006) supra; Kireeva, et al. (2002) supra). Non-permissive 603R nucleosomes present a high, polar barrier to Pol II progression (Bondarenko, et al. (2006) supra). DNA fragments bearing single positioned nucleosomes were ligated downstream of pre-assembled transcript elongation complexes EC−119 (the numerical indices indicate the position of the Pol II active center on the template relative to promoter-proximal nucleosomal DNA boundary of the nucleosome). Then nascent RNA was pulse-labeled by forming EC−83 and transcription was resumed in the presence of an excess of unlabeled NTPs (Kireeva, et al. (2002) supra). The fraction of Pol II molecules that reach the end of the template (run-off) was used to quantify the height of the barrier, which is located primarily in the +45 region.

Mutations introduced into the critical HA sequences (603R-R template) resulted in much higher fraction of templates transcribed to completion, as compared with the 603R template (65% and 32% at 300 mM KCl, respectively). Sixty-five percent corresponds to the upper limit of read-through efficiency by Pol II achieved on the permissive templates or templates devoid of HA sequences (Bondarenko, et al. (2006) supra; Kireeva, et al. (2002) supra). Thus, the -R mutations convert the non-permissive 603R template into the permissive 603R-R template. In contrast, the transcriptional properties of the 603R and 603R-L templates are nearly identical. To determine whether the observed decrease in the height of the barrier was caused by a change in nucleosome positioning on the 603R-R template, the 603R-R(2-3) nucleosomal template was transcribed. The mutations in the -R(2-3) sequences resulted in strong relief of the barrier without affecting nucleosome positioning. Thus, the high affinity of the -R(2-3) sequences for histones dictated a strong nucleosomal barrier to transcription.

In summary, these experiments indicate that, surprisingly, the critical DNA sequences that confer the high nucleosomal barrier to Pol II transcription (the HA sequences) are located more than 40 bp downstream of the active center of the enzyme arrested at the +45 region of the nucleosome.

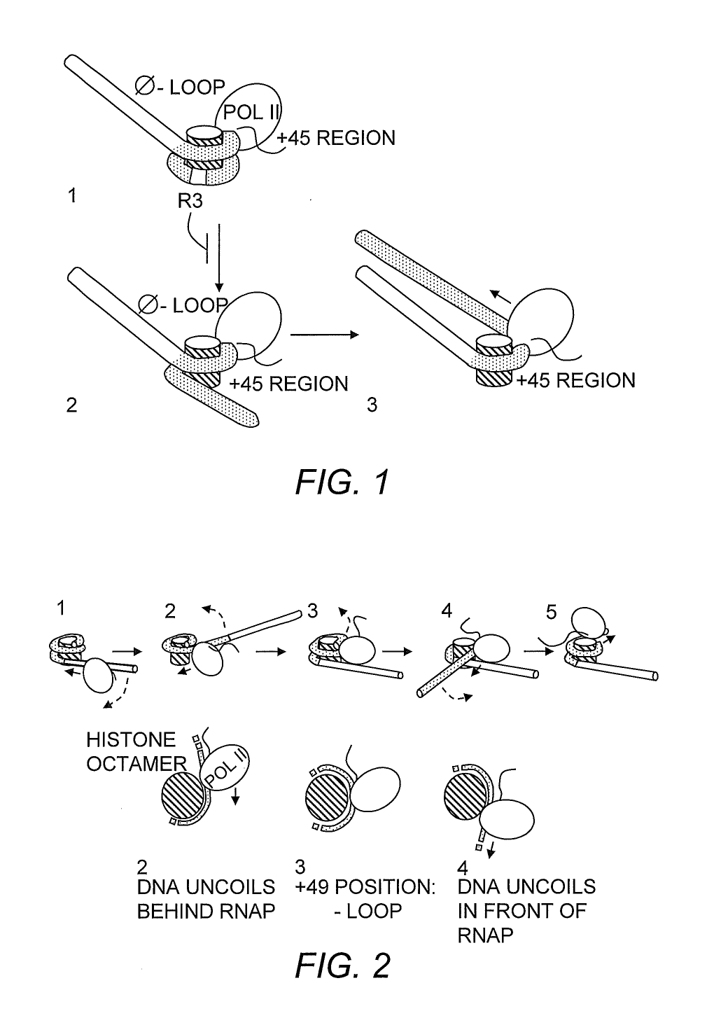

Modeling of Pol II Elongation Complexes in a Nucleosome. It was subsequently determined how DNA sequences located far downstream of Pol II can induce arrest in the +45 region of the nucleosome. One possibility was that nucleosomal structure was involved in formation of the barrier. Previous studies have suggested that, during productive transcription, Pol II localized at the +45 region induces uncoiling of nucleosomal DNA from the octamer to allow further transcription (Bondarenko, et al. (2006) supra). Therefore, it was determined whether Pol II-induced DNA uncoiling could explain the action of the downstream HA sequences over a distance. It was contemplated that as the Pol II molecule transcribes through the +45 region, it can form a tight intranucleosomal DNA “zero-size” loop (Ø-loop) containing the active enzyme (FIG. 1, (1)). Formation of the Ø-loop would result in steric interference between Pol II and the promoter-distal end of the nucleosomal DNA. This, in turn, could induce partial uncoiling of DNA from the octamer ahead of Pol II and facilitate further progression through a permissive nucleosome (FIG. 1, (2) and (3)). Conversely, downstream HA sequences could prevent DNA uncoiling and thus hinder further transcription through a non-permissive nucleosome. Similar uncoiling of promoter-distal DNA end from the octamer and formation of the Ø-loop were observed in studies of the single-subunit, non-homologous bacteriophage SP6 RNAP stalled at the +45 region (Bednar, et al. (1999) Mol. Cell 4:377-386).

To evaluate the possibility of Ø-loop formation by the 12-subunit Pol II, the Ø-loop was modeled by docking the high-resolution structures of yeast Pol II elongation complex (EC) and the nucleosome (Luger, et al. (1997) Nature 389:251-260; Kettenberger, et al. (2004) supra). This analysis indicated that formation of a Ø-loop was sterically possible when Pol II was at the position +39 or +49 in a nucleosome and at least 50 bp were displaced from the promoter-distal end of nucleosomal DNA.

Modeling of the Ø-loop-containing EC+39 indicated that the bulk of the Pol II molecule faced into solution and there were no steric clashes with core histones. In addition, the 90° DNA bend present in the EC faced the octamer surface and allowed formation of the Ø-loop. Furthermore, an extensive octamer surface was available to establish contacts with ˜20-bp DNA region behind the EC and formed the Ø-loop. Formation of the Ø-loop and displacement of by from the promoter-distal end of the nucleosome reduced the size of the DNA region interacting with histones in front of the enzyme from ˜100 to ≦50 bp. This would facilitate further uncoiling of DNA from the octamer ahead of Pol II and transcription through the nucleosome. Moreover, the R3 HA DNA sequence (CTAGA; SEQ ID NO:8) was located within the displaced 50-bp DNA region such that the R3 HA sequences would be expected to interfere with DNA displacement and trigger Pol II arrest in the +45 region. The steric constraints revealed during the modeling were not sufficiently strict to allow reliable identification of the potential interacting side chains that form the Pol II-nucleosome interface in EC+39. However, the model allowed evaluation of the potential interacting surfaces in the complex. In particular, the modeling identified a negatively charged region on the surface of Pol II that may be important for proper transcription through chromatin. This region may form electrostatic interactions with the histone octamer in EC+39 and/or EC+49, thereby stabilizing the complex.

Formation of the Ø-loop was possible only in one rotational orientation of the EC on DNA (at positions +39 or +49). Movement of the enzyme by 1 nucleotide would result in a ˜36° rotation around the DNA axis and steric clashes between Pol II and the histone octamer. Thus, Pol II translocation after formation of the Ø-loop would disrupt the DNA-histone interactions upstream and/or downstream of the enzyme. If only the downstream histone-DNA interactions are broken (FIG. 1, (3)), Pol II could transcribe through chromatin without complete displacement of the octamer into solution, as has been observed experimentally (Kireeva, et al. (2002) supra).

As indicated, the steric constraints revealed during modeling of the EC+39 were not sufficiently strict to allow reliable identification of the potential interacting side chains that form Pol II-nucleosome interface in the EC+39. To further evaluate the potential interacting surfaces in the complex, charge distribution on the Pol II-nucleosome interface (PDB IDs 1aoi and 1y1w; Luger, et al. (1997) Nature 389:251-260; Kettenberger, et al. (2004) Mol. Cell 16:955-965) was analyzed. This analysis revealed a strong negative charge on the surface of Pol II in close proximity to positively charged region on the surface of the histone octamer. These regions are located within the clamp core domain of RPB1 subunit of Pol II and most likely form electrostatic interactions within the EC+39. The same positively charged region on the surface of the histone octamer interacts with DNA in the original nucleosome; these interactions are disrupted in the EC+39. Therefore, the electrostatic Pol II-histone interactions within the EC+39 may compensate for the DNA-histone interactions that are disrupted during formation of the elongation complex, and thus stabilize the EC+39. Similar interactions may stabilize the EC+49 complex. If the negatively charged region on the surface of Pol II is important for proper transcription through chromatin, this region is expected to be conserved between E. coli RNAP and Pol II that transcribe through chromatin using similar mechanisms. Sequence comparison of the relevant negatively charged regions of the β′ (E. coli RNAP) and RPB1 (S. cerevisiae Pol II) subunits showed 39% sequence identity and 59% sequence similarity. In particular, 5 out of 6 critical negatively charged residues were preserved and one residue was replaced by a polar amino acid. More than 50% of the net negative charge of the conserved region was preserved in E. coli RNAP. Taken together, the data indicate that the negatively charged region on the surface of Pol II may be important for proper transcription through chromatin. It is likely that this region forms electrostatic interactions with the histone octamer in EC+39 and/or EC+49 complexes to compensate for the absence of contacts of the basic histone residues with the phosphate backbone of displaced DNA and thus stabilizes these critical complexes.

Formation of an Intranucleosomal Ø-Loop. The proposed model for transcription through chromatin by Pol II (FIG. 1) was evaluated by footprinting of ECs stalled at various positions within permissive 603 mononucleosomes. It was extremely technically challenging to obtain large quantities of homogeneous Pol II ECs stalled at a desired position in a nucleosome. All general aspects of the Pol II-type mechanism were recapitulated by E. coli RNAP (Walter, et al. (2003) supra) but not by other previously analyzed RNAPses (Bondarenko, et al. (2006) supra; Studitsky, et al. (1994) supra; Studitsky, et al. (1997) supra). Since homogeneous E. coli ECs can be obtained in sufficient amounts, they were used for the initial analysis of the Pol II-type mechanism of transcription through chromatin. The results of this analysis indicated that both the characteristic pausing patterns (including the strong barrier in the +45 region) and the relative overall efficiencies of transcription of permissive and non-permissive nucleosomes (Bondarenko, et al. (2006) supra) were recapitulated using E. coli RNAP.

Modeling indicated that the Ø-loop could be formed when RNAP transcribed 39 or 49 bp of nucleosomal DNA. Therefore, a subset of ECs halted near these positions on the 603 template was analyzed. To obtain homogeneous stalled ECs, uniquely positioned nucleosomes were pre-assembled, transcription was initiated, and the ECs were “walked” to the desired positions within nucleosomal DNA after incubation with different subsets of NTPs (positions −39, −5, +42 or +49).

To map the position of the active center of RNAP in the ECs, they were incubated in the presence of GreB. GreB strongly facilitates RNA cleavage by E. coli RNAP; the cleavage reaction is mediated by the RNAP active site and occurs only in complexes formed after backtracking of the enzyme along the DNA and RNA chains (Laptenko, et al. (2003) EMBO J. 22:6322-6334; Borukhov, et al. (1993) Cell 72:459-466; Komissarova & Kashlev (1997) J. Biol. Chem. 272:15329-15338). In backtracked (paused or arrested) complexes, the nascent RNA is rapidly shortened by several nucleotides in the presence of GreB, reporting on the extent of RNAP reverse translocation. It was found that EC+49 complexes were resistant to GreB, indicating that the active center remained associated with the 3′-end of the RNA. When stalled at +42, RNAP backtracked by 1-2 nucleotides to form EC+41, but was not arrested.

The structures of these complexes were analyzed using single-hit digestion with DNA endonuclease (DNaseI). This analysis indicated that each stalled EC (e.g., EC−39) protected ˜30-bp, and the nucleosome protected ˜150-bp from DNaseI digestion. When RNAP formed EC+41, the nucleosomal DNA was completely uncoiled from the octamer upstream of the RNAP. The DNA downstream of the ECs remained fully bound but was distorted around the +90 and +100 positions, as shown by the appearance of hypersensitive sites.

Although the EC+49 was stalled inside the nucleosome, the nucleosome-specific features of the footprint persisted on the majority (≧70%) of the complexes, indicating that the 603 nucleosome remained at its original position with DNA fully wrapped around the octamer. In this complex, DNA protection by the EC was not easily discernible, likely because the nucleosome-specific DNA protection masked protection by the EC. However, it was contemplated that the EC+49 should remain active during the 30-second digestion with DNaseI, because most complexes produced run-off transcripts. The persistence of the nucleosome-specific DNA protection pattern in EC+49 indicated that the original DNA-histone contacts were re-formed both upstream and downstream of the stalled RNAP. This was possible only if the EC+49 formed the Ø-loop on the surface of the histone octamer. Therefore, as indicated by the model (FIG. 1), RNAP stalled after transcription of 49 bp of 603 nucleosomal DNA forms the Ø-loop.

Although the nucleosome-specific features predominate in EC+49, DNA both upstream and downstream of the EC was more accessible to DNaseI than in the original nucleosome. Quantitative analysis revealed that the accessibility of nucleosomal DNA upstream of RNAP (+15 to +25 region) was less pronounced than it was downstream of the enzyme. Further, a short DNA region at the promoter-proximal end of the nucleosomal DNA (+1 to +20) was almost completely resistant to DNase I and multiple DNA sites downstream of EC+49 were accessible to DNaseI to a similar degree, but considerably less than in histone-free DNA. Most likely, both upstream and downstream contacts were lost in the same ternary complex because otherwise it is difficult to explain how the contacts between +20 and +35 can be disrupted without displacing the +1 to +20 region at the end of nucleosomal DNA. Together, these data indicate that DNA is uncoiled from the octamer in front of the enzyme on ≦30% of templates (forming an “open” intermediate) and on an even smaller (≦10%) fraction of templates, the nucleosomal DNA is partially uncoiled from the octamer upstream of RNAP. The +1 to +20 region remains fully associated with the octamer. Since the histone octamer is not lost, the intermediates are most likely in rapid equilibrium, with the majority of the complexes being in the closed conformation.

Taken together, these data indicate a pathway for productive transcription through a permissive nucleosome (FIG. 2). As RNAP enters the nucleosome, it initially uncoils nucleosomal DNA primarily behind itself, as seen in EC+41. As the enzyme reaches the +49 position, the DNA behind RNAP is re-coiled on the surface of the octamer, the Ø-loop is formed, and the DNA in front of the complex becomes partially uncoiled from the octamer (EC+49). Sequential release of DNA-histone contacts in the Ø-loop intermediate allows both unimpeded transcription (through selective disruption of the downstream interactions) and nucleosome recovery (through re-formation of the original DNA-histone interactions upstream of RNAP).

To confirm that E. coli RNAP and Pol II form similar key complexes during transcription through a nucleosome, Pol II was stalled at positions −5, +42 or +49 in the 603 nucleosome and the structures of the complexes were analyzed using a Cac8I and StyI restriction enzyme sensitivity assay. Nucleosomes strongly protect DNA from digestion with restriction enzymes (Polach & Widom (1999) Methods Enzymol. 304:278-298); thus both the Cac8I and StyI intranucleosomal sites (positions +15 and +79, respectively) are protected from digestion in EC−5 and in intact nucleosomes. The results of this analysis indicated that in EC+41, the DNA behind Pol II (at +15, the Cac8I site) was sensitive to digestion and the DNA in front of the enzyme (at +79, the StyI site) was resistant. Importantly, the StyI site is located far downstream of the Pol II boundary on the DNA (Gnatt, et al. (2001) Science 292:1876-1882). In contrast, in EC+49, the Cac8I site was largely protected and the StyI site was accessible. These data indicate that Pol II and RNAP induced similar structural rearrangements of DNA/histone contacts during transcription through 603 nucleosomes. Together with previous observations indicating that nucleosomes survive at the original position following Pol II passage (Kireeva, et al. (2002) supra), the results herein indicate that the Ø-loop is formed when Pol II reaches the position +49. Then the loop is resolved in front of the enzyme, and transcription continues.

In summary, the data indicate that the structures of the intermediates formed before and after Pol II reaches the position +49 are very different: initially nucleosomal DNA is displaced upstream of Pol II, but distal to position +49, DNA displacement occurs primarily downstream of the enzyme (FIG. 2). Thus, formation of the Ø-loop at the +49 position constitutes the transition point during transcription through a nucleosome that allows nucleosome recovery at the original position on the DNA.

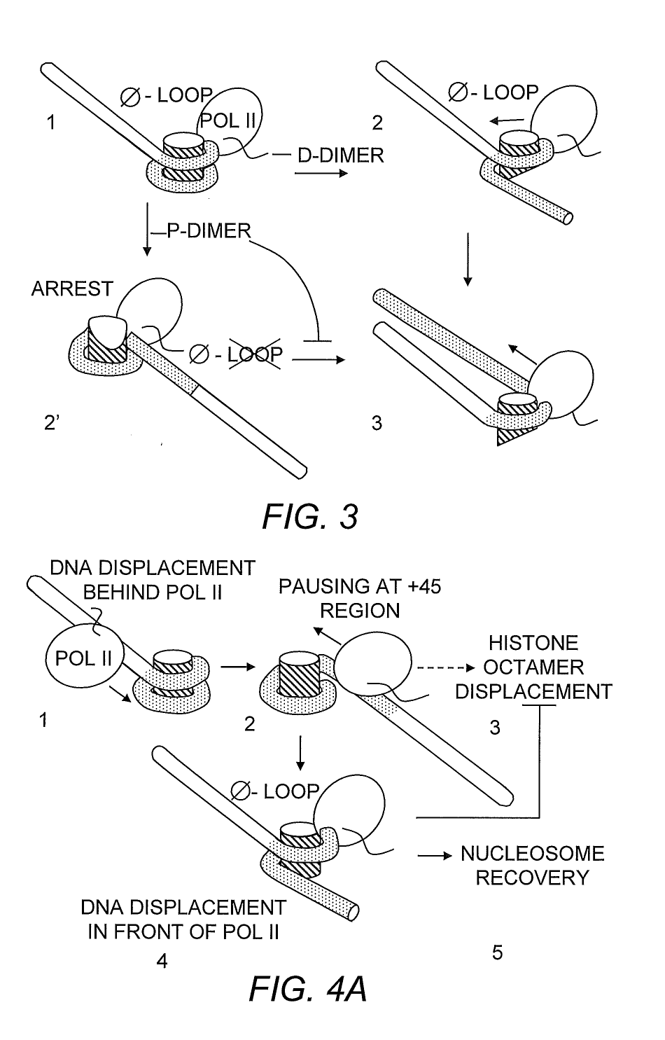

H2A/H2B Dimers have Opposite Effects on the Nucleosomal Barrier to Transcription. The asymmetric roles of the histone-DNA contacts are inherent in the model described herein (FIG. 2). Removal of the distal histone H2A/H2B dimer (D-dimer) would result in release of the promoter-distal end of nucleosomal DNA into solution, facilitating formation of the Ø-loop (FIG. 3, (1) and (2)) and therefore would result in facilitated transcription through the nucleosome (FIG. 3, (3)). In contrast, removal of the proximal P-dimer would result in disruption of DNA-histone contacts upstream of the EC and would strongly destabilize the Ø-loop (FIG. 3, (2′)). In the latter case, two scenarios are possible: (a) If the upstream contacts are not essential for further transcription, Pol II would displace the DNA immediately downstream and continue. (b) Alternatively, if the upstream contacts are essential, their disruption by removal of the P-dimer would cause nucleosome-specific arrest at the +45 region. Thus, the model indicates that removal of the promoter-proximal or the promoter-distal dimer could have drastically different impacts on the height of the +45 nucleosomal barrier (FIG. 3).

To evaluate the latter possibility, permissive 603 nucleosomes and subnucleosomes missing either the P- or the D-dimer (-P- and -D-hexasomes, respectively) were constructed and transcribed by Pol II. The results of this analysis indicated that removal of the promoter-distal D-dimer resulted in a partial relief of the +45 barrier. Since the +45 barrier in the permissive orientation is not very strong (Bondarenko, et al. (2006) supra), this relief does not have a strong impact on the yield of the run-off transcript.

In sharp contrast, removal of the promoter-proximal P-dimer resulted in a strong increase (8- and 12-fold at 40 and 150 mM KCl, respectively) in the strength of the +45 barrier. Therefore, the DNA-histone contacts upstream of the EC paused at the +45 region were essential for further transcription through the nucleosome. The strength of the +45 barrier in permissive 603 nucleosomes missing the P-dimer approaches the one characteristic of non-permissive nucleosomes (Bondarenko, et al. (2006) supra). Thus, removal of the P-dimer transforms the permissive nucleosome into a non-permissive one.

Consistent with model described herein (FIG. 2), the P- and D-dimers play very different roles within the same nucleosome. Removal of the P-dimer resulted in a strong Pol II arrest in the +45 region, most likely because the Ø-loop could not form, the promoter-distal end of nucleosomal DNA could not be displaced, and transcription was hindered (FIG. 3). In contrast, removal of the D-dimer resulted in a partial relief of the +45 barrier, most likely because displacement of the promoter-distal end of the nucleosomal DNA and formation of the Ø-loop were facilitated by removal of this dimer.

Implications of Conformational Changes in Nucleosomal Structure During Transcription. Structural analysis of the elongation complexes formed during transcription through a nucleosome indicates a new mechanism of transcription through chromatin (FIG. 2). This mechanism is consistent with the strong effects of far-downstream sequences on Pol II pausing in the +45 intranucleosomal DNA region (FIG. 1) and the requirement of the promoter-proximal histone H2A-H2B dimer for efficient Pol II transcription through the nucleosome (FIG. 3).

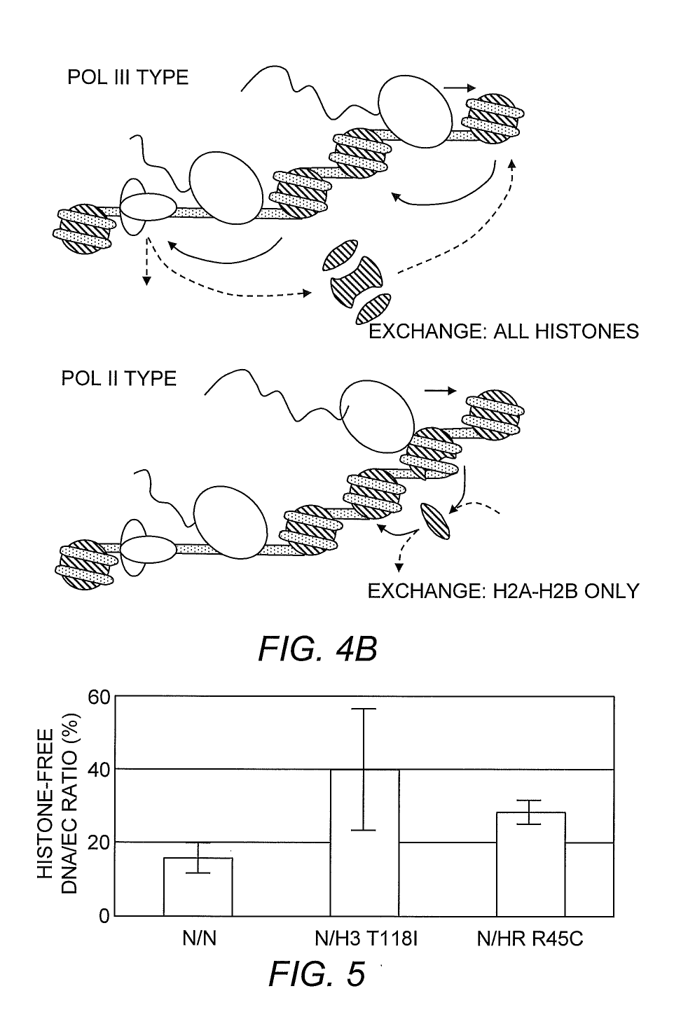

This mechanism operates on various DNA sequences and relies on a feedback loop to couple nucleosome survival to efficient transcription through chromatin via conformational changes in the nucleosome structure (FIG. 4A), wherein if a nucleosome cannot survive transcription, Pol II becomes arrested. Because the key features of the process of transcription through a nucleosome by yeast and human Pol II are highly similar (Bondarenko, et al. (2006) supra), the mechanism may well be conserved in all eukaryotes.

The differences in the structures of the intermediates formed by the Pol II- and Pol III-type mechanisms could explain the different nucleosome fates during these processes. At the same time, the Ø-loop can be formed during transcription through the +45 region by both mechanisms (Luger, et al. (1997) supra). These observations indicate that the conformational dynamics involved in formation of the Ø-loop are intrinsic to nucleosomes. Therefore, the conformational changes in nucleosomal structure observed during transcription are likely to occur during progression of other processive enzymes (for example, ATP-dependent remodelers and DNA polymerases) through chromatin.

Many eukaryotic genes are regulated at the level of transcript elongation, and nucleosomes may be key players in this regulation (Mavrich, et al. (2008) Nature 453:358-362; Core, et al. (2008) Science 322:1845-1848; Formosa, et al. (2002) Genetics 162:1557-1571; Pavri, et al. (2006) Cell 125:703-717). The observations herein indicate that inefficient resolution of the Ø-loop can cause strong nucleosome-specific pausing, and the paused intermediate could be a target for regulation of the rate of elongation through chromatin. Such regulation could be mediated by variable promoter-distal sequence of nucleosomal DNA and/or by histone chaperones (for example, FACT; Kireeva, et al. (2002) supra; Belotserkovskaya, et al. (2003) supra; van Holde, et al. (1992) J. Biol. Chem. 267:2837-2840; Formosa, et al. (2002) Genetics 162:1557-1571; Pavrl, et al. (2006) Cell 125:703-717) facilitating displacement of the distal D-dimer.

In contrast, the proximal P-dimer is likely to be essential for both nucleosome survival and efficient transcription. Displacement of the P-dimer leads to arrest of Pol II within the nucleosome (FIG. 3) and most likely occurs only transiently in vivo. Because both H2A-H2B dimers are exchanged during Pol II transcription in vivo (Thiriet & Hayes (2005) supra), the P-dimer must be immediately replaced to avoid arrest of transcribing Pol II, loss of the histone octamer or both. Therefore, the data herein indicate that a transcription-coupled process could guarantee fast rebinding of the displaced H2A-H2B dimer(s) to nucleosomes. Factors facilitating P-dimer rebinding are expected to facilitate transcription through chromatin. Indeed, the H2A-H2B chaperone FACT associates with elongating Pol II, contributes to nucleosome survival during transcription in vivo (Belotserkovskaya, et al. (2003) supra; Formosa, et al. (2002) supra; Pavrl, et al. (2006) supra) and facilitates transcription through nucleosomes in vitro (Bondarenko, et al. (2006) supra; Belotserkovskaya, et al. (2003) supra).

Recent studies suggest that on moderately Pol II-transcribed genes the exchange of H3 and H4 histones is at least 20-fold slower than that of H2A-H2B. The results herein indicate that H3 and H4 are not exchanged because they are never completely misplaced from the DNA during Ø-loop-mediated transcription through chromatin. Because most eukaryotic genes are transcribed at moderate levels, transcription-dependent exchange of bulk H3 and H4 histones is considerably slower than exchange of H2A-H2B dimer (Kimura & Cook (2001) J. Cell Biol. 153:1341-53).

Pol II transcription through chromatin is coupled with nucleosome survival at the original position on DNA. Nucleosome survival at the original position is important because transcription of a eukaryotic gene using the alternative, Pol III-type mechanism would trigger an extensive displacement and exchange of all core histones (Studitsky, et al. (1994) Cell 76:371-283). On the contrary, a Pol II-type mechanism involves only minimal exchange of histones H3 and H4 (FIG. 4B). Because the H3 and H4 histones contain the majority of the sites for post-translational modifications, including some epigenetic marks (Kouzarides (2007) Cell 128:693-705), the Pol II-type mechanism specifically allows for the survival of the original H3 and H4 histones and their covalent modifications during transcription. Because almost the entire eukaryotic genome is transcribed at a certain non-zero frequency (David, et al. (2006) Proc. Natl. Acad. Sci. USA 103:5320-5325; Araki, et al. (2006) Stem Cells 24:2522-2528), this mechanism would mediate maintenance of epigenetic marks across the genome.

### Example 2

### Histone Sin Mutations Promote Nucleosome Traversal and Histone Displacement by RNA Polymerase II

Methods. Preparation of proteins, nucleosome reconstitution by salt dialysis, assembly of transcription complexes and transcription of nucleosomal templates were performed as described in Example 1; Bondarenko, et al. (2006) supra; and Újvári, et al. (2008) J. Biol. Chem. 283:32236-32243. Briefly, yeast pol II complexes were assembled from RNA, template and non-template DNAs; they were then immobilized on Ni-NTA beads, washed, eluted and ligated to nucleosomes. After pulse-labeling of 45 mer RNA, complexes were chased into the nucleosomes with excess non-labeled NTPs. For transcription assays, complexes were resolved by native polyacrylamide gel electrophoresis (PAGE) after transcription. For human pol II, nucleosomes were reconstituted on template DNAs and bound to beads, followed by assembly of pre-initiation complexes with pol II and transcript initiation factors. After pulse-labeling of 21 mer RNA and rinsing, transcripts were chased into the nucleosomes with excess non-labeled NTPs. Salt concentrations and additions to reactions are as described for each experiment. Labeled RNAs were resolved by denaturing PAGE and quantified using a PHOSPHORIMAGER.

Transcription by E. coli RNAP and DNase footprinting were carried out as described in Example 1. In brief, nucleosomes were reconstituted on end-labeled 147 bp DNA, followed by ligation of the T7A1 promoter upstream. Transcription by RNAP was stalled 41 bp in the nucleosome, followed by treatment with DNase I for 30 seconds at 37° C. DNase-digested complexes were resolved by native PAGE. DNA was extracted from the complexes, separated by denaturing PAGE and quantified using a PHOSPHORIMAGER.

For the experiments of this Example, yeast or human pol II transcribed a template bearing a single downstream nucleosome assembled at a precise location (Bondarenko, et al. (2006) supra; and Újvári, et al. (2008) supra). Transcripts were labeled during an initial pulse, followed by a rinse and chase with excess non-labeled NTPs. The two nucleosome assembly elements used in this study were designated 603 (Lowary & Widom (1998) J. Mol. Biol. 276:19-42), which is more permissive for traversal, and 603R, which is less permissive (Újvári, et al. (2008) supra). Sin mutations affect gene expression to different extents (Kruger, et al. (1995) Genes Dev. 9:2770-2779). Yeast Sin mutations have been incorporated into analogous locations in Xenopus H3 or H4 and the structures and physical properties of nucleosomes containing the mutant histones determined (Muthurajan, et al. (2004) EMBO J. 23:260-271). H3 T118I and H4 R45C showed the greatest effects in thermal mobilization studies of nucleosomes, whereas H3 R116H and H4 V43I had lesser effects. The recombinant Xenopus histones were used in the instant experiments.

Sin Mutations Reduce the Nucleosomal Barrier to Transcription. Nucleosomes were assembled on the 603 or 603R template using wild-type or tailless H2A/H2B, and wild-type or Sin mutant H3 and H4. RNA in yeast polymerase II complexes was initially pulse-labeled and then extended at KCl concentrations of 40, 150, and 300 mM or 1% sarkosyl with excess unlabeled NTPs. The locations of the nucleosome, the nucleosome dyad, the main pol II pause sites and the position of the run-off transcript were determined. The results of this analysis indicated that traversal of wild-type 603 nucleosomes by either yeast or human pol II was highly inefficient at 40 mM salt and increased only slightly at 150 mM KCl. Prominent stops were observed at +15 and in the +45 region.

The 603 transcription barrier was reduced significantly when either H3 T118I or H4 R45C was incorporated. Pausing was primarily lowered at or near +45. By contrast, traversal of 603 nucleosomes with H3 R116H histones was almost unchanged from that of wild-type. Similarly, for yeast pol II, no increase in traversal was observed after substitution of H4 V43I for wild-type H4. Thus, the extent of relief of the 603 nucleosomal barrier to pol II transcription by various Sin mutations seemed to be correlated with their effect on thermal mobilization of nucleosomes (Muthurajan, et al. (2004) supra).

Nucleosomes assembled on the 603R template provide a consistently stronger barrier to transcript elongation in comparison with 603 nucleosomes (Bondarenko, et al. (2006) supra). Yeast pol II was not used for the 603R studies, as the +45 region on this template represents a strong barrier to transcript elongation for the yeast enzyme as pure DNA. Substitution of either H3 T118I or H4 R45C reduced the pausing by human pol II at +45 and strongly reduced (R45C) or eliminated (T118I) the +55 pause. Traversal of the 603R nucleosomes with H3 T118I by human pol II increased about three-fold relative to wild-type at both 40 and 150 mM KCl. Interestingly, the barrier relief relative to wild-type provided by H4 R45C was distinctly lower than the barrier reduction with H3 T1181; further, barrier relief for human pol II on 603R was comparable for H3 R116H and H4 R45C, in contrast to the results with 603 nucleosomes. In preliminary tests, the barrier reduction for human pol II on the 603R template provided by incorporation of the H4 V43I mutant was also comparable to the reduction obtained with the H4 R45C mutant. The effects of Sin mutations on human pol II traversal efficiency were clearly not identical on all templates. As Sin mutations affect mostly the +45/+55 barrier, the differences between the effects of different mutations on traversal could be more evident on the non-permissive templates.

Sin Mutations and Removal of Histone Tails Reduce the Transcriptional Barrier by Different Mechanisms. Removal of some or all of the N-terminal histone tails can also result in significant decreases in the barrier to complete nucleosomal traversal by both yeast and human pol II Újvári, et al. (2008) supra). To determine whether the Sin mutations and tail removal reduce the nucleosome traversal barrier by a common mechanism, nucleosomes assembled with tailless H2A and H2B and wild-type H3/H4 tetramers, or tetramers containing Sin mutants H3 T118I or H4 R45C were tested. A significant reduction in the traversal barrier was expected from the removal of only the H2A/H2B tails (Újvári, et al. (2008) supra). Incorporation of tailless H2A/H2B further reduced the nucleosomal barrier for human pol II with the Sin mutant histones. Similar effects were observed for yeast pol II on the 603 template with tailless H2A/H2B and each of the four Sin mutations. Importantly, relief of the 603 traversal barrier by tail removal and by incorporation of H3 T118I and H4 R45C Sin mutations seemed to occur by independent mechanisms. As shown in Table 2, the percentage increase in traversal on 603 templates conferred by the incorporation of both a Sin mutant histone and tailless H2A/H2B was either roughly equal to or greater than the sum of the increases provided by either nucleosome alteration alone.

TABLE 2

| | % Traversal | | | |
|---|---|---|---|---|
| | | n/Sin | | |
| Reactants | n/n | Mutant | g/n | g/Sin mutant |
| H3 T118I, 603: | | | | |
| Yeast, 40 mM KCl | 7 | 19 (+12) | 18 (+11) | 37 (+30; +23) |
| Yeast, 150 mM KCl | 35 | 72 (+37) | 51 (+16) | 85 (+50; +53) |
| Human, 40 mM KCl | 19 | 37 (+18) | 26 (+7) | 60 (+42; +25) |
| Human, 150 mM KCl | 54 | 74 (+20) | 65 (+11) | 85 (+31; +31) |
| H4 R45C, 603: | | | | |
| Yeast, 40 mM KCl | 7 | 17 (+10) | 18 (+11) | 30 (+23; +21) |
| Yeast, 150 mM KCl | 35 | 66 (+31) | 51 (+16) | 80 (+45; +47) |
| Human, 40 mM KCl | 19 | 35 (+17) | 26 (+8) | 49 (+30; +25) |
| Human, 150 mM KCl | 54 | 72 (+19) | 65 (+11) | 81 (+27; +30) |
| H3 T118I, 603R: | | | | |
| Human, 40 mM KCl | 13 | 41 (+28) | 24 (+11) | 56 (+43; +39) |
| Human, 150 mM KCl | 25 | 85 (+60) | 46 (+21) | 79 (+54; +81) |
| H4 R45C, 603R: | | | | |
| Human, 40 mM KCl | 13 | 23 (+10) | 24 (+11) | 41 (+28; +21) |
| Human, 150 mM KCl | 25 | 51 (+27) | 46 (+21) | 63 (+38; +48) |

For each combination of pol II, KCl concentration, template and Sin mutant, four values are given: the percent traversal with all wild-type histones (n/n), with the indicated Sin mutant H3 or H4 (n/Sin mutant), with tailless H2A/H2B (g/n) or with both tailless H2A/H2B and the indicated Sin mutant (g/Sin mutant). Traversal was set to 100% for reactions containing 1M KCl (yeast pol II) or 1% sarkosyl (human pol II). For the n/Sin mutant and g/n tests, the value in parenthesis next to the traversal level (+x) gives the traversal increase in that case relative to n/n. For the g/Sin mutant tests, two values are given in parenthesis: first, the traversal increase relative to n/n; second (underlined), the sum of the increases from incorporation of only the Sin mutant or only the tailless histones.

The effect of tail removal on traversal of 603R Sin mutant nucleosomes by human pol II at 150 mM KCl was less straightforward. In particular, tail removal provided no additional stimulation at 150 mM KCl in the context of nucleosomes containing the T118I H3 variant. However, the T118I mutation alone relieved almost all the transcriptional barrier on 603R at 150 mM KCl; hence, there was little opportunity for further barrier reduction in that case.

The TFIIS transcript elongation factor stimulates traversal of nucleosomal 603 templates in the presence or absence of the histone N-terminal tails (Újvári, et al. (2008) supra). It was found that yeast TFIIS simulated traversal of wild-type and Sin mutant 603 nucleosomal templates by 2-2.5-fold, in the presence or absence of the H2A/H2B N-terminal tails. Relief of the 603 nucleosomal barrier by tail removal, as well as by Sin mutations and TFIIS, appeared to occur by independent mechanisms.

Sin Mutations Increase the Likelihood of Histone Displacement During Pol II Transcription. Sin mutations affect the histone-DNA interactions in the region from +70 to +80 of nucleosomal DNA that make a significant contribution to overall nucleosome stability (Muthurajan, et al. (2004) supra; Hall, et al. (2009) Nat. Struct. Mol. Biol. 16:124-129). Therefore, it was determined whether the Sin mutant nucleosomes were more likely to dissociate from the template on traversal by pol II. DNA-labeled nucleosomes were transcribed by yeast pol II and analyzed in a native gel to monitor the histone-free DNA generated. The amount of DNA released from Sin nucleosomes was larger than the amount released from intact nucleosomes (FIG. 5). In the case of the H3 T118I mutant at 150 mM KCl, the octamer was displaced from ˜40% of templates. These data indicate that interactions between H3/H4 histones with the region from +70 to +80 of nucleosomal DNA are important for nucleosome integrity and survival during transcript elongation by pol II.

Sin Mutation-Induced Histone Displacement can be Detected when RNA Polymerase Reaches Position +41. Sin mutations reduce the major +45 pause by pol II; they also increase complete nucleosomal traversal and decrease nucleosomal survival on traversal. Given that Sin mutations reduce crucial DNA-octamer interactions across the segment of nucleosomal DNA from +70 to +80, uncoiling of downstream DNA from the octamer surface should be more probable when RNA polymerase arrives at the major pause site on Sin mutant nucleosomes. DNase I footprinting can be used to test this, but to obtain interpretable footprints it is essential to analyze nearly homogeneous transcript elongation complexes. Even under optimal conditions, a substantial fraction of templates fail to be transcribed by pol II; in addition, it is not possible to advance pol II by any distance into a nucleosome without stalling many complexes at various upstream positions. Thus, it was necessary to perform the footprinting studies with Escherichia coli RNA polymerase (RNAP). Results with RNAP are relevant to pol II transcription because pol II and RNAP share the same mechanism of nucleosome traversal (Walter, et al. (2003) J. Biol. Chem. 278:36148-36156). To further confirm the similarities of the mechanisms of transcription through the 603 nucleosome by RNAP and pol II, wild-type and H3 T118I Sin mutant nucleosomes were transcribed by RNAP at 150 mM KCl. The results of this analysis indicated that Sin mutant nucleosomes showed a reduction in +45 and +55 pauses and an increase in nucleosome traversal relative to wild-type nucleosomes.

An RNAP EC stalled at +41 in the 603 nucleosome was selected for the footprinting studies because it was shown in Example 1 that DNA-histone interactions are maintained in front of RNAP in this nucleosomal transcription complex when wild-type histones are used for nucleosome assembly. EC+41 complexes stalled on pure 603 DNA or nucleosomal templates were digested with DNase I under single-hit conditions. When the bacterial polymerase was stalled at +41 on the nucleosome assembled with wild-type histones, nucleosomal DNA was accessible to DNase I digestion upstream from the RNAP, but the DNA downstream remained inaccessible. By contrast, the nucleosomal DNA both upstream and downstream from the EC+41 RNAP was accessible to DNase I in the Sin nucleosome. These results are fully consistent with the observations that (i) Sin mutant nucleosomes provide a significantly reduced transcriptional barrier at +45 for pol II and (ii) traversal of Sin mutant nucleosomes by pol II is more likely to result in complete dissociation of the template DNA from the histone octamer.

Overall, the results of this analysis indicated that Sin mutations reduce the barrier to nucleosome traversal by pol II and increase the probability of nucleosome loss on transcription. Sin mutant nucleosomes may have these properties because Sin mutations further destabilize an intermediate that already has a limited number of DNA-protein interactions. As described in Example 1, a crucial intermediate called a zero (Ø)-loop complex can form when pol II swings away from the octamer surface at +39. As pol II approaches the nucleosome, it initially displaces the promoter-proximal DNA, which subsequently begins to reassociate with the octamer surface, forming the +39 Ø-loop. At the same time, uncoiling of the downstream template is facilitated by a steric clash between the advancing polymerase and downstream DNA. It is expected that, on wild-type nucleosomes, strong histone-DNA interactions flanking the nucleosome dyad prevent full uncoiling of the downstream DNA, resulting in a high probability of histone survival. However, on nucleosomes with Sin mutant histones, critical dyad-proximal interactions are weaker. This should further destabilize the +39 complex, favoring the full unwinding of the downstream DNA. Note that DNA-histone association is already weakened in an RNAP complex stalled at +41 in a Sin mutant nucleosome. An increased probability of unwinding facilitates traversal, but also increases the probability that the nucleosome will be displaced during transcription.

On the 603 nucleosome, barrier reduction from the removal of the H2A/H2B tails is additive with the reduction obtained with the Sin mutant histones. Loss of H2A/H2B tails reduces the second strongest histone-DNA interactions, located 25-35 bp from each nucleosome boundary (Brower-Toland, et al. (2005) J. Mol. Biol. 346:135-146; Hall, et al. (2009) supra). Consistent with this, the major barrier at +15 on the 603 nucleosome is reduced specifically in the absence of H2A/H2B tails. However, H2A/H2B tail removal also causes a significant increase in nucleosome traversal on the 603R template by human pol II, where there is no +15 pause. In this case, H2A/H2B tail removal is presumably disrupting the interaction 25-35 bp from the downstream edge of the nucleosome (the region from +112 to +122), which should also facilitate partial displacement of the DNA end from the octamer surface during the complex transition. In summary, different segments of the histone-DNA interface are expected to be disrupted by Sin mutations and tail removal, which would explain the generally additive stimulation of traversal caused by these two alterations in nucleosome structure.

What is claimed is:

1. A method for identifying an agent that modulates the RNA Polymerase II-histone surface comprising
contacting a nucleosome with a test agent in the presence of a RNA Polymerase II elongation complex and
determining whether the agent modulates resolution of a Ø-loop formed by the nucleosome and RNA Polymerase II elongation complex thereby identifying an agent that modulates the RNA Polymerase II-histone surface.

2. An agent identified by the method of claim 1.

3. The agent of claim 2, wherein said agent stabilizes the RNA Polymerase II-histone surface.

4. The agent of claim 2, wherein said agent destabilizes the RNA Polymerase II-histone surface.

5. A method for preventing or treating a disease caused by improper maintenance of histones, and modifications thereof, comprising administering to a subject in need thereof an effective amount of an agent that stabilizes RNA Polymerase II-histone surfaces.

6. A method for preventing or treating a disease caused by over efficient maintenance of histones, and modifications thereof, comprising administering to a subject in need thereof an effective amount of an agent that destabilizes RNA Polymerase II-histone surfaces.


SEQUENCE LISTING (8 sequences)

SEQ ID NO: 1 - DNA, 71 bp, Artificial sequence
  Synthetic oligonucleotide
gagtaatccccttggcggttaaaacgcgggggacagcgcgtacgtgcgtttaagcggtgctagagctgtct

SEQ ID NO: 2 - DNA, 71 bp, Artificial sequence
  Synthetic oligonucletodie
ttcaacatcgatgcacggtggttagccttggattgcgctctaccgtgcgctaagcgtacttagaagcccga

SEQ ID NO: 3 - DNA, 71 bp, Artificial sequence
  Synthetic oligonucleotide
tgaatacccttgggcggctaaaacgacggggctagggttgtaacgtcgtttaagcgtatctagaccggtct

SEQ ID NO: 4 - DNA, 71 bp, Artificial sequence
  Synthetic oligonucleotide
annnnnntctagnnnngcttaaanngnnnnanangncntntnnnncnntttaagcnnnnctagannnnnnt

SEQ ID NO: 5 - DNA, 28 bp, Artificial sequence
  Synthetic oligonucleotide
tcgatgcacggtggttagccttggattg

SEQ ID NO: 6 - DNA, 26 bp, Artificial sequence
  Synthetic oligonucleotide
tctaccgtgcgctaagcgtacttaga

SEQ ID NO: 7 - DNA, 14 bp, Artificial sequence
  Synthetic oligonucleotide
taagcgtacttaga

SEQ ID NO: 8 - DNA, 5 bp, Artificial sequence
  Synthetic oligonucleotide
ctaga